(19) United States
(12) Patent Application Publication
(10) Pub. No.: US 2002/0114812 A1
(43) Pub. Date: Aug. 22, 2002

(54) Methods and compositions for modulating regulation of the cytotoxic lymphocyte response by macrophage migration inhibitory factor

(76) Inventors: Riichiro Abe, Kita-Ku (JP); Richard Bucala, Cos Cob, CT (US); Christine N. Metz, Great Neck, NY (US)

Correspondence Address: Supervisor, Patent Prosecution Services, PIPER MARBURY RUDNICK & WOLFE LLP, 1200 Nineteenth Street, N.W., Washington, DC, 20036-2412, US

(21) Appl. No.: 10/043,322

(22) Filed: Jan. 14, 2002

Related U.S. Application Data
(60) Provisional application No. 60/260,914, filed on Jan. 12, 2001.

(51) Int. Cl. A61K 39/395; A61K 48/00

(52) U.S. Cl. 424/155.1; 514/44; 424/93.21

(57) ABSTRACT

Regulation of expression of CTL activity by macrophage migration inhibitory factor (MIF) is disclosed. In a mouse model using the EL4 tumor, cultured splenocytes from tumor-primed mice secrete high levels of MIF following antigen stimulation in vitro. Parallel splenocytes treated with neutralizing anti-MIF mAb showed a significant increase in CTL response against tumor cells compared to control mAb-treated cultures, with elevated expression of IFNγ. Histology of tumors from anti-MIF treated animals showed increases in infiltration of both CD4⁺ and CD8⁺ T cells, as well as apoptotic tumor cells, consistent with observed augmentation of CTL activity in vivo by anti-MIF, which was associated with enhanced expression of the common γ_{c} chain of the IL-2 receptor that mediates CD8⁺T cell survival. CD8⁺ cells of anti-MIF treated tumor-bearing mice showed increased migration into tumors of control mice. Methods for enhancing a CTL response by inhibition of MIF are disclosed.

### BACKGROUND OF THE INVENTION

1. Field of the Invention

The present invention relates to methods and compositions for modulating (increasing or decreasing) a cytotoxic lymphocyte response to an antigen, such as a tumor-associated antigen, by decreasing or increasing the level of macrophage migration inhibitory factor (MIF) to which CD8⁺ and/or CD4⁺ lymphocytes are exposed before, during or after exposure to the antigen. The invention further relates to compositions and methods for prophylaxis and treatment of diseases, particularly tumors, by modulating a cytotoxic lymphocyte response to an antigen using cell-based immunotherapeutic approaches.

2. Background of the Technology

Emerging data from both experimental and human clinical studies indicate that tumor associated antigens are sufficient to elicit an anti-tumor cytotoxic lymphocyte (CTL) response that can produce significant tumor regression (27, 28). Long-term melanoma remissions have been achieved in a few cases by employing cell-based immunotherapeutic strategies aimed at enhancing CTL cytotoxicity by peptide immunization (29). However, despite the presence of tumor-specific antigens presented in the context of MHC class I, robust tumor killing immune response is seldom detected in vivo. The generation of tumor-specific CTLs requires appropriate processing of tumor antigens, display of tumor antigens by MHC class I molecules, T lymphocytes expressing T cell receptors of appropriate specificity to recognize tumor antigens, and initial antigen presentation to the immune system in an immunologic context. This CTL response must not only be initiated, but must also be vigorous and be sustained to achieve successful tumor regression.

The activity of several cytokines to enhance various aspects of the CTL response has been appreciated for some time. The early expression of IL-2 for example, is a critical factor in the proliferation and development of lytic potential by CTLs (30). Furthermore, IFNγ (30), IL-1 and IL-6 (31), IL-2 together with IL-6 (32), IL-7 (33), IL-10 (34), and IL-12 (35-37) have all been identified to play a role in the activation, proliferation, and/or differentiation of CTLs. These mediators promote CTL activity by enhancing antigen presentation, CD4⁺ helper T cell function, macrophage cell adhesion, or by increasing the expression of critical co-stimulatory molecules. Anti-tumor effects mediated by the administration of recombinant cytokines, including IL-1 (38), IL-2 (39), IL-12 (40-42), IFNα (43, 44), IFNγ (45), and TNFα (46) have been shown in tumor bearing-mice.

By contrast, only a few cytokines, including IL-4 (47, 48) and TGFβ (49) have been shown to suppress CTL differentiation or lytic activity. IL-4 inhibits the secretion of IFNγ from CD8⁺ T cells (50, 51) and appears to limit the activation and differentiation of CD8⁺ T cells with high cytolytic potential (52). Furthermore, CTL priming in the absence of IL-4 gives rise to a more potent response following challenge. The mechanisms by which these few cytokines inhibit CTL cytolytic activity are not well defined.

The biological functions of the protein mediator known as macrophage migration inhibitory factor (MIF) have only recently come under close scrutiny (reviewed in (1)). Although MIF was first described nearly four decades ago as a soluble activity produced by activated T lymphocytes (2, 3), interest in MIF was rekindled when the mouse homolog of this protein was identified to be secreted from the anterior pituitary gland (4). Soon thereafter macrophages that had been previously considered to be a target of MIF action were found to be a significant source of MIF upon activation by microbial toxins or the cytokines TNFα and IFNγ (5). In vivo studies also established that MIF plays a critical role in the host response to endotoxin. Administration of recombinant MIF (rMIF) together with LPS exacerbates LPS lethality, while neutralizing anti-MIF antibodies protect mice against lethal endotoxemia (4), exotoxemia (6), and peritonitis (7). Studies of MIF function also have established this protein to be required for the expression of IL-2 during the T-cell activation response and for antibody production by B cells (8).

Two recent reports have identified an unanticipated role for MIF in tumor growth (9, 10). The present inventors observed that the administration of an anti-MIF monoclonal antibody (mAb) to mice significantly reduced the growth and vascularization of the syngeneic, subcutaneously implanted B cell lymphoma, 38C13 (9). Evidence was obtained that this anti-tumor effect was due, in part, to a requirement for MIF in endothelial cell proliferation and the tumor angiogenesis response (9). Similarly, anti-MIF mAb treatment of mice bearing the human melanoma tumor, G361, significantly decreased tumor growth and neovascularization (10).

### SUMMARY OF THE INVENTION

The instant invention is based, in part, on the discovery by the present inventors that MIF expression is upregulated during the CTL response and that inhibition of MIF using a specific mAb promotes CTL activity in vitro and in vivo. In particular, disclosed herein is experimental evidence that neutralization of MIF can promote CTL activity, inhibit tumor growth, and increase T lymphocyte homing to sites of tumor invasion in vivo. Thus, results from in vitro CTL studies in the Example, below, reveal that immunoneutralization of MIF during the in vitro priming phase increased IFNγ production in CTL cultures. Recognizing that MIF secretion is enhanced by the activation of Th2 cells, but not Th1 cells (8), it is possible that soluble antigen stimulation induces MIF expression that in turn inhibits CTL activation in vivo by suppressing the production of Th1 cytokines, including IFNγ.

Previous studies have shown that MIF plays an essential role in the activation response to various mitogens or soluble antigen, an effect that is mediated by CD4⁺ helper T cells. Mitogen or antigen-activated T cells express significant quantities of MIF mRNA and protein, and immunoneutralization of MIF inhibits IL-2 production and T cell proliferation in vitro and decreases the T cell helper response to soluble antigen in vivo (8). The present study shows that MIF expression is upregulated in response to tumor antigen stimulation and that neutralization of MIF does not affect IL-2 secretion or antigen-induced proliferation of CD8⁺ T cells. However, anti-MIF treatment significantly increased the expression of the IL-2 receptor γ_{c} subunit that is required for intracellular signaling (25) and is important for CD8⁺ T cell survival (26). Therefore, the enhancement of T cell cytotoxicity by MIF neutralization cannot be attributed to an appreciable increase in the proliferation of CD8⁺ T cells, but rather to enhanced survival of a population of cytolytic CD8⁺ T cells. Following the initiation of cytolytic activity by CD8⁺ T cells, this cytolytic activity must be sustained in order to promote successful tumor regression. Accordingly, inhibition of MIF would act to prolong CTL lifespan such that significant CTL anti-tumor activity becomes manifest both in vitro and in vivo.

Anti-MIF mAb treatment of EG.7 tumor-bearing mice significantly inhibited tumor growth in the context of enhanced CTL activity. Moreover, CD8⁺ T cells transferred from anti-MIF treated anti-MIF treated tumor-bearing mice inhibited tumor growth in recipient mice. Given the observed increase in the number of apoptotic tumor cells found within the corpus of the tumor, it is reasonable to conclude that enhanced or sustained CTL cytotoxicity directly contributed to the suppression of tumor growth in anti-MIF treated mice.

Recent reports have shown that tumor cells produce more MIF than non-transformed cells (10, 53, 54). Tumor cells can escape death by CTLs via the loss of the tumor antigen recognized by the CTLs or by the downregulation of MHC expression that renders the tumor cell resistant to CTL-mediated lysis even when it expresses the appropriate tumor antigen (55). Although EG.7 cells constitutively secrete MIF (˜10 ng/ml by 10⁶ cells), neither rMIF nor anti-MIF antibody influenced MHC class I expression by EG.7 cells. The present data show that an additional mechanism for tumor evasion of the host immune response occurs by tumor cell secretion of MIF leading to a decrease in CD8⁺ T cell survival.

Several studies have shown the expression of FasL by some tumor cells and this raises the intriguing possibility that cancers might be sites of immune privilege. For example, apoptosis of tumor infiltrating lymphocytes has been demonstrated in situ in FasL-expressing melanomas and hepatocellular carcinomas (57). However, more recent in vitro and in vivo data have challenged the original hypothesis. These studies have revealed that some tumors lack FasL expression (58, 59) and that transfection of some tumor cells with FasL cDNA did not promote evasion of the immune system by tumor cells, but rather induced tumor regression (59, 60). Further studies have shown that FasL expression promotes rapid graft rejection (61, 62) and inflammation (63). This study did not examine the expression of FasL within the tumor, but the present findings show that it would be informative to examine the effect of MIF/anti-MIF on FasL expression in these systems.

The present study has also identified an important role for MIF in T cell trafficking. An increase in the accumulation of both CD4⁺ and CD8⁺ T cells within the tumors of anti-MIF treated mice was observed. Tumor destruction by tumor infiltrating lymphocytes (TILs) is known to involve both CD4⁺ and CD8⁺ T cells. Treatment of breast tumors in rats with IL-2 and TILs promotes tumor regression by the induction of apoptosis in the tumor cells (64) and a brisk accumulation of TILs in human melanoma is associated with a more favorable outcome for the patient (65). The observation that anti-MIF antibody increases the migration of CD4⁺ and CD8⁺ T cells into the tumor mass provides an additional means by which anti-MIF antibody may affect anti-tumor T cell function, and may involve mechanisms such as altered chemokine or chemokine receptor expression.

In addition to modulating CTL activity, MIF appears to play a role in other aspects of tumor formation. Two independent laboratories have shown that MIF neutralization significantly inhibits tumor angiogenesis (9, 10), and Hudson and co-workers recently revealed that the addition of rMIF to fibroblasts inhibits p53 functions (both proliferation and apoptosis) by suppressing its transcriptional activity (66). Although a variety of host immune effector cells participate in the killing of tumor cells, tumor antigen-specific CTLs are highly effective in mediating tumor cell killing, even at low antigen density expressed on the target cells (67). Hence, the therapeutic enhancement of CD8⁺ CTLs by MIF immunoneutralization provides a novel basis for cell-based anti-tumor immunotherapies.

Accordingly, the present invention provides methods and compositions for modulating (increasing or decreasing) a cytotoxic lymphocyte response to an antigen, such as a tumor-associated antigen, by decreasing or increasing the level of macrophage migration inhibitory factor (MIF) to which CD8⁺ and/or CD4⁺ lymphocytes are exposed before, during or after exposure to the antigen, either ex vivo or in vivo, or both.

Thus, in one aspect the present invention provides a method of preparing cells, preferably T cells, more preferably CD8⁺ T cells, as a cancer therapy for administration to a subject with cancer or another condition requiring a CTL response for effective immunotherapy. This method comprises culturing the cells in the presence of an MIF antagonist or inhibitor. In this method, the MIF antagonist is selected from the group consisting of anti-MIF antibodies, MIF antisense cDNA, and antagonists of MIF ligand:receptor binding. In a preferred embodiment of this method comprises culturing the cells in the presence of anti-MIF antibodies that neutralize or inactivate MIF activity. Preferably, the anti-MIF antibodies used in the invention method are monoclonal and are selected from the group consisting of human monoclonal antibodies, humanized monoclonal antibodies, chimeric monoclonal antibodies and single-chain monoclonal antibodies.

In another aspect the present invention relates to a method of preparing a cellular composition as an immunotherapy for enhancing a CTL response, preferably a cancer therapy for administration to a subject with cancer, comprising incubating cells of the composition in the presence of (a) at least one antigen that is a target of a desired CTL response, preferably a tumor antigen, and (b) anti-MIF antibodies.

Yet another aspect of the invention relates to a method of preparing autologous cells for administration to a subject with cancer comprising the step of incubating the cells in the presence of an agent, agent selected from the group consisting of anti-MIF antibodies, MIF-binding fragments thereof, or both. A preferred embodiment of this method comprises a step of incubating the cells in the presence of (a) at least one tumor antigen and (b) an agent selected from the group consisting of anti-MIF antibodies, MIF-binding fragments thereof, or both. Preferably, the autologous cells comprise immune cells, more preferably T cells, and even more preferably, CD8⁺ T cells.

In another aspect the invention provides a cellular composition for administration to a subject in need of an enhance CTL response to an antigen, for instance, a subject with cancer. This composition comprises cells incubated with anti-MIF antibodies. In one embodiment of this cellular composition, the cells incubated with anti-MIF antibodies are also incubated with at least one antigen to which an enhanced CTL response is desired, such as a tumor antigen. Preferably, in this cellular composition the incubation with anti-MIF antibodies is ex vivo, and the cellular composition may include cells isolated from unbound anti-MIF antibodies after incubation with anti-MIF antibodies. Cells in this cellular composition also may be isolated from both unbound anti-MIF antibodies and unbound antigen, for instance, tumor antigen, with which they are incubated. Preferably, in the cellular compositions of the invention the cells comprise immune cells, more preferably T cells, and still more preferably, CD8⁺ T cells.

### DESCRIPTION OF THE FIGURES

FIG. 1.—Anti-MIF mAb, but not rMIF or control IgG, enhances CTL activity in vitro. C57BL/6 mice primed with EG.7 cells 7 days earlier were the source of spleen cells (see Materials and Methods). Spleen cell cultures stimulated with irradiated EG.7 cells for 5 days in the presence of rMIF (A), anti-MIF (B), or control IgG₁, (C). Fresh EG.7 target cells were added to spleen cells at various E:T cell ratios and, after a 4 h incubation at 37° C., cytotoxicity measured by lactate dehydrogenase (LDH) release. (D) The effect of anti-MIF mAb on in vitro CTL activity upon antibody addition at the onset of splenocyte-irradiated EG. 7 co-cultures (E:T=20:1) (Day 0) versus addition at Day 2. *, p<0.05 by Student's t test vs. no addition.

FIG. 2—Secretion of MIF and IFNγ is enhanced when primed spleen cells are cultured with irradiated EG.7 cells. Spleen cells were isolated from EG.7-primed mice and stimulated for 1 or 2 days with or without irradiated EG.7 cells together with an isotype control Ab (control) or anti-MIF mAb (anti-MIF) (50 μLg/ml). Culture supernatants were analyzed by specific ELISA for MIF(A) and IFNγ(B), as described in Materials and Methods. TNFα and IL-12 values were below the limit of detection. *, p<0.05 by Student's t test for control+EG.7 vs. control-E.7.

FIG. 3—Anti-MIF mAb treatment of EG.7 tumor-bearing mice increases CTL activity and inhibits tumor growth. C57BL/6 mice (n=5 per group) were injected with EG.7 cells and then treated with either PBS, control IgG, or anti-MIF mAb (0.5 mg) daily. On day 7, the spleens were harvested and isolated spleen cells were co-cultured with irradiated EG.7 cells for 5 days, at which time cell lysis was measured in a 4 h CTL in vitro assay by LDH release (A). Tumor size was determined on Day 7 (B). *, p<0.05 by Student's t test anti-MIF treated vs. control IgG treated.

FIG. 4—Anti-MIF mAb treatment of EG.7 tumor-bearing mice increases T lymphocyte infiltration of tumors. Mice (n=5 per group) were treated daily for 7 days with control IgG or anti-MIF mAb. Then, EG.7 tumors were excised and tumor sections were stained with PE-anti-mouse CD4 (L3T4) or FITC-anti-mouse CD8 (ly-2) monoclonal antibodies. CD8⁺ and CD4⁺ T cells were enumerated by fluorescence microscopy and expressed as the average percent increase (±S.D.) in immunoreactive infiltrating cells in the tumors of anti-MIF-treated animals compared to control IgG-treated animals. Sections incubated with a fluorescent-isotype control antibody showed no immunoreactivity. *, p<0.05 by Student's t test comparing anti-MIEF vs control IgG treated.

FIG. 5—Anti-MIF mAb treatment promotes EG.7 tumor cell apoptosis. Apoptotic cells were detected in situ by labeling DNA strand breaks by the TUNEL method. Numerous apoptotic (dark brown) EG.7 cells are visible in the tumor tissue obtained from mice treated with anti-MIF mAb (A). By contrast, fewer apoptotic bodies are observed in tumors obtained from mice treated with control IgG (B). Sections (100×) shown are representative of 10 tumor sections (n=5 animals per group).

FIG. 6—IL-2Rγ_{c} expression is upregulated by treatment with anti-MIF antibody in vivo. Spleen cells were collected from naive or EG.7 tumor-bearing mice (n=3 mice per group) treated daily for 7 days with anti-MIF or control IgG. Spleens were pooled from individual groups and stained for CD8 and IL-2Rα (A), β (B), or γ_{c} (C) surface markers after gating on the CD8⁺ T cell population. The shaded histogram represents the cells stained with isotype control antibody.

FIG. 7—Treatment of donor tumor-bearing mice with anti-MIF antibody increases the migration of transferred T lymphocytes into EG.7 tumors of recipient tumor-bearing mice and promotes CD8⁺ T cell anti-tumor activity in recipient mice. Unfractionated spleen cells (A) or purified CD8⁺ splenic T cells (B) from normal (control) or tumor-bearing mice were isolated 8 days after anti-MIF or control IgG treatment (0.5 mg×7 days) and labeled with the fluorescent dye, PKH-26. Labeled cells then were transferred i.v. into tumor-bearing recipient mice (n=5 per group). One day later, the tumors were excised, cryostat sections prepared, and the number of fluorescent cells per high power field (HPF) was enumerated (mean±S.D.). *, p<0.05; **, p<0.01 vs. control antibody treated mice by Student's t test. (C) C57BL/6 mice were injected with 5×10⁶ EG.7 cells (i.p.) and then treated with anti-MIF mAb or control IgG (0.5 mg/day) for 7 days. Purified splenic CD8⁺ T cells were transferred (5×10⁶ cells/mouse; i.v). into recipient mice that had been inoculated s.c. with 5×10⁶ EG.7 cells 24 h previously (n=5 per group). Tumor weights (average ±S.D.) were measured. *, p<0.05 vs control antibody-treated mice by Student's t test.

### DETAILED DESCRIPTION OF THE INVENTION

The present invention involves compositions and methods that inhibit MIF release and/or activity in vitro and in vivo, for the treatment of any conditions requiring a CTL response, which include but are not limited to tumors (cancerous or benign), viral infections, parasitic infections, including for instance malaria, and/or bacterial infections.

The inhibition of MIF activity in accordance with the invention may be accomplished in a number of ways, which may include, but are not limited to, the use of factors which bind to MIF and neutralize its biological activity; the use of MIF-receptor antagonists; the use of compounds that inhibit the release of MIF from cellular sources in the body; and the use of nucleotide sequences derived from MIF coding, non-coding, and/or regulatory sequences to prevent or reduce MIF expression. Any of the foregoing may be utilized individually or in combination to inhibit MIF activity in the treatment of the relevant conditions, and further, may be combined with any other CTL enhancing therapies including, for instance, peptide immunization, cytokine therapy, and the like.

Factors that bind MIF and neutralize its biological activity, hereinafter referred to as MIF binding partners, may be used in accordance with the invention as treatments of conditions requiring a CTL response. While levels of MIF protein may increase due to secretion by a tumor or by activation of Th2 T helper cells, the interaction of inhibitory MIF-binding partners with MIF protein prohibits a concomitant increase in MIF activity. Such factors may include, but are not limited to anti-MIF antibodies, antibody fragments, MIF receptors, and MIF receptor fragments.

Various procedures known in the art may be used for the production of antibodies to epitopes of recombinantly produced (e.g., using recombinant DNA techniques described infra), or naturally purified MIF. Neutralizing antibodies, i.e. those which compete for or sterically obstruct the binding sites of the MIF receptor are especially preferred for diagnostics and therapeutics. Such antibodies include but are not limited to polyclonal, monoclonal, humanized monoclonal, chimeric, single chain, Fab fragments and fragments produced by an Fab expression library.

For the production of antibodies, various host animals may be immunized by injection with MIF and/or a portion of MIF. Such host animals may include but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Monoclonal antibodies to MIF may be prepared by using any technique which provides for the production of antibody molecules by continuous cell lines in culture. These include but are not limited to the hybridoma technique originally described by Kohler and Milstein, (Nature, 1975, 256:495-497), the human B-cell hybridoma technique (Kosbor et al., 1983, Immunology Today, 4:72; Cote et al., 1983, Proc. Natl. Acad. Sci., 80:2026-2030) and the EBV-hybridoma technique (Cole et al., 1985, Monoclonal Antibodies and Cancer Therapy, Alan R. Liss, Inc., pp. 77-96). In addition, techniques developed for the production of “chimeric antibodies” (Morrison et al., 1984, Proc. Natl. Acad. Sci., 81:6851-6855; Neuberger et al., 1984, Nature, 312:604-608; Takeda et al., 1985, Nature, 314:452-454) by splicing the genes from a mouse antibody molecule of appropriate antigen specificity together with genes from a human antibody molecule of appropriate biological activity can be used. Alternatively, techniques described for the production of single chain antibodies (U.S. Pat. No. 4,946,778) can be adapted to produce MIF-specific single chain antibodies.

The hybridoma technique has been utilized to generate anti-MIF monoclonal antibodies. See, e.g., U.S. Pat. No. 6,030,615 to Bucala et al., the entire contents of which are hereby incorporated herein by reference. Hybridomas secreting IgG monoclonal antibodies directed against both human and murine forms of MIF have been isolated and characterized for their ability to neutralize MIF biological activity. Anti-MIF monoclonal antibodies were shown to inhibit the stimulation of macrophage-killing of intracellular parasites. The anti-MIF monoclonal antibodies have also been utilized to develop a specific and sensitive ELISA screening assay for MIF.

Antibody fragments which recognize specific MIF epitopes may be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al., 1989, Science, 246:1275-1281) to allow rapid and easy identification of monoclonal Fab fragments with the desired specificity to MIF.

MIF receptors, MIF receptor fragments, and/or MIF receptor analogs may, in accordance with the invention, be used as inhibitors of MIF biological activity. By binding to MIF protein, these classes of molecules may inhibit the binding of MIF to cellular MIF receptors, thus disrupting the mechanism by which MIF exerts its biological activity. Small organic molecules which mimic the activity of such molecules are also within the scope of the present invention. MIF receptors may include any cell surface molecule that binds MIF in an amino acid sequence-specific and/or structurally-specific fashion. Fragments of MIF receptors may also be used as MIF inhibitory agents, and any MIF receptor fragment possessing any amino, carboxy, and/or internal deletion that specifically binds MIF so as to inhibit MIF biological activity is intended to be within the scope of this invention. An amino and/or carboxy deletion refers to a molecule possessing amino and/or carboxy terminal truncations of at least one amino acid residue. An internal deletion refers to molecules that possess one or more non-terminal deletions of at least one amino acid residue. Among these MIF receptor fragments are truncated receptors in which the cytoplasmic or a portion of the cytoplasmic domain has been deleted, and fragments in which the cytoplasmic and the transmembrane domain(s) has been deleted to yield a soluble MIF receptor containing all or part of the MIF receptor extracellular domain.

MIF receptor analogs which specifically bind MIF may also be used to inhibit MIF activity. Such MIF receptor analogs may include MIF receptor or receptor fragments further possessing one or more additional amino acids located at the amino terminus, carboxy terminus, or between any two adjacent MIF receptor amino acid residues. The additional amino acids may be part of a heterologous peptide functionally attached to all or a portion of the MIF receptor protein to form a MIF receptor fusion protein. For example, and not by way of limitation, the MIF receptor, or a truncated portion thereof, can be engineered as a fusion protein with a desired Fc portion of an immunoglobulin. MIF receptor analogs may also include MIF receptor or MIF receptor fragments further possessing one or more amino acid substitutions of a conservative or non-conservative nature. Conservative amino acid substitutions consist of replacing one or more amino acids with amino acids of similar charge, size, and/or hydrophobicity characteristics, such as, for example, a glutamic acid (E) to aspartic acid (D) amino acid substitution. Non-conservative substitutions consist of replacing one or more amino acids with amino acids possessing dissimilar charge, size, and/or hydrophobicity characteristics, such as, for example, a glutamic acid (E) to valine (V) substitution. The MIF receptors, MIF receptor fragments and/or analogs may be made using recombinant DNA techniques.

Molecules which inhibit MIF biological activity by binding to MIF receptors may also be utilized for the treatment of conditions requiring a CTL response. Such molecules may include, but are not limited to anti-MIF receptor antibodies and MIF analogs. Anti-MIF receptor antibodies may be raised and used to neutralize MIF receptor function. Antibodies against all or any portion of a MIF receptor protein may be produced, for example, according to the techniques described in U.S. Pat. No. 6,080,407, supra.

MIF analogs may include molecules that bind the MIF receptor but do not exhibit biological activity. Such analogs compete with MIF for binding to the MIF receptor, and, therefore, when used in vivo, may act to block the effects of MIF in the progress of cytokine-mediated toxicity. A variety of techniques well known to those of skill in the art may be used to design MIF analogs. Recombinant DNA techniques may be used to produce modified MIF proteins containing, for example, amino acid insertions, deletions and/or substitutions which yield MIF analogs with receptor binding capabilities, but no biological activity. Alternatively, MIF analogs may be synthesized using chemical methods (see, for example, Sambrook et al., Molecular Cloning, A Laboratory Manual, Cold Spring Harbor Press, N.Y. (1989)). MIF receptors and/or cell lines that express MIF receptors may be used to identify and/or assay potential MIF antagonists.

As taught in U.S. Pat. No. 6,080,407, supra. certain steroids, commonly thought to be either inactive or “anti-steroidal” actually inhibit the release of MIF; e.g., 20α dihydrocortisol. These steroids, or any other compound which inhibits the release of preformed MIF, can be used in combination therapy with other anti-MIF agents in the present invention.

Inhibitors of MIF biological activity such as anti-MIF antibodies, MIF receptors, MIF receptor fragments, MIF receptor analogs, anti-MIF receptor antibodies, MIF analogs and inhibitors of MIF release, may be administered using techniques well known to those in the art. Preferably, agents are formulated and administered systemically. Techniques for formulation and administration may be found in “Remington's Pharmaceutical Sciences”, 18th ed., 1990, Mack Publishing Co., Easton, Pa. Suitable routes may include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections, just to name a few. Most preferably, administration is intravenous. For injection, the agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

Effective concentrations and frequencies of dosages of the MIF inhibitory compounds invention to be administered may be determined through procedures well known to those in the art, which address such parameters as biological half-life, bioavailability, and toxicity. In the case of anti-MIF antibodies, a preferred dosage concentration may range from about 0.1 mg/kg body weight to about 20 mg/kg body weight, with about 10 mg/kg body weight being most preferred. For antibodies or other inhibitory compounds that exhibit long half-lives in circulation, a single administration may be sufficient to maintain the required circulating concentration. In the case of compounds exhibiting shorter half-lives, multiple doses may be necessary to establish and maintain the requisite concentration in circulation.

MIF inhibitors may be administered to patients alone or in combination with other therapies. Such therapies include the sequential or concurrent administration of inhibitors or antagonists of tumors or viruses for which a CTL response is desirable.

Within the scope of the invention are methods using anti-MIF agents that are oligoribonucleotide sequences, including anti-sense RNA and DNA molecules and ribozymes that function to inhibit the translation of MIF and/or MIF receptor mRNA. Anti-sense RNA and DNA molecules act to directly block the translation of mRNA by binding to targeted mRNA and preventing protein translation, either by inhibition of ribosome binding and/or translocation or by bringing about the nuclease degradation of the mRNA molecule itself. Ribozymes are enzymatic RNA molecules capable of catalyzing the specific cleavage of RNA. The mechanism of ribozyme action involves sequence specific hybridization of the ribozyme molecule to complementary target RNA, followed by a endonucleolytic cleavage. Within the scope of the invention are engineered hammerhead motif ribozyme molecules that specifically and efficiently catalyze endonucleolytic cleavage of MIF and/or MIF receptor mRNA sequences. Both anti-sense RNA and DNA molecules and ribozymes of the invention may be prepared by any method known in the art for the synthesis of nucleic acid molecules. These include techniques for chemically synthesizing oligodeoxyribonucleotides well known in the art such as, for example, solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various modifications to the DNA molecules may be introduced as a means of increasing intracellular stability and half-life. Possible modifications include but are not limited to the addition of flanking sequences of ribo- or deoxy-nucleotides to the 5′ and/or 3′ ends of the molecule or the use of phosphorothioate or 2′ O-methyl rather than phosphodiesterase linkages within the oligodeoxyribonucleotide backbone.

For anti-MIF therapeutic uses, the inhibitory oligonucleotides may be formulated and used with cells in vitro, and/or administered through a variety of means, including systemic, and localized, or topical, administration. Techniques for formulation and administration may be found in “Remington's Pharmaceutical Sciences,” Mack Publishing Co., Easton, Pa., latest edition. The mode of administration may be selected to maximize delivery to a desired target organ in the body.

### EXAMPLE

Materials and Methods

Experimental animals and cell lines. C57BL/6 (H-2b) mice (female, 8-12 wk old) were purchased from The Jackson Laboratory (Bar Harbor, Me.). All animal procedures were conducted according to guidelines of the NSUH Institutional Animal Care and Use Committee under an approved protocol. EG.7 cells (produced by transfection of EL4 with a cDNA encoding OVA (11)) and EL4 cells (both MHC class II negative, H-2^{b} murine thymomas), as well as the YAC-1 cells were obtained from ATCC (Rockville, N. Mex.).

Cytokines and antibodies. Recombinant murine MIF (rMIF) was prepared as previously described (12;13) (<I pg endotoxin/μg protein). Neutralizing anti-MIF mAb (clone XIV. 15.5, IgG₁, isotype) was prepared as previously described (9, 14). An isotype control antibody (IgG₁) was purified under similar conditions using the hybridoma, 5D4-11, which secretes antibody specific for type 3 dengue virus (ATCC). FITC-rat anti-mouse CD3 Ab, PE-rat anti-mouse CD4, PerCP-rat anti-mouse CD8 Ab, PE-rat anti-mouse CD25 Ab, PE-rat anti-mouse CD28 Ab, FITC-rat anti-mouse CD44 Ab, PE-rat anti-mouse CD25 (IL-2Rα), PE-rat anti-mouse CD28 Ab, FITC-rat antimouse CD44 Ab, PE-rat anti-mouse CD25 (IL-2Rα), PE-rat anti-CD122 (IL-2Rβ), PE-rat anti-mouse CD132 (shared γ chain), and PE-rat anti-mouse H-2K^{b} were purchased from PharMingen (San Diego, Calif.).

Generation of antigen-specific CTL. The generation of OVA-specific CTL has been described previously (11). In brief, spleen cells were obtained from mice primed 1-2 weeks earlier by i.p. injection of 5×10⁶ EG.7 cells. Isolated spleen cells (3×10⁶) were incubated with irradiated EG.7 cells (20,000 rad; 10⁶ cells) for five days (in the presence or absence of cytokines or antibodies—see below). Effector cells used in the in vitro CTL assay (see below) were collected from these cultures and recognized the OVA₂₅₇₋₂₆₄ (SIINFKEL) peptide in the context of H-2K^{b} (15). To study the effect of MIF neutralization in vivo, EG.7-primed mice received an injection of anti-MIF mAb or control IgG (0.5 mg, i.p.) on the day of tumor cell implantation and then daily for 1 week. Spleen cells from anti-MIF or control IgG treated mice then were isolated and assessed for CTL activity in vitro as described below.

Cell-mediated cytotoxity assay. EG.7 target cells (5×10⁵/well) were added to serial dilutions of effector spleen cells (prepared as described above) in 96-well round bottom plates at E:T ratios of 1:1 to 30:1 together with various concentrations of anti-MIF mAb, control IgG, or purified rMIF. After 4 h at 37° C., cytotoxicity was quantified by measurement of the cytosolic enzyme, lactate dehydrogenase (LDH) in the culture supernatant (n=3) using the CytoTox 96° Assay (Promega Madison, Wis.). ‘Specific lysis’ for each E:T ratio is expressed as: specific lysis=[(experimental release)−(spontaneous release)/(target maximum−target spontaneous release)]. Spontaneous LDH release in the absence of CTL was less than 10% of the maximal cellular release by detergent lysis. All experimental procedures and assays were performed two or more times, with similar results.

NK assay. NK sensitive YAC-1 cells were used as targets and NK assays were performed as previously described (16).

Flow cytometric analysis. Single-cell suspensions free of erythrocytes were prepared from the pleens of experimental mice as indicated and analyzed by flow cytometry. All fluorescently labeled ntibodies were purchased from PharMingen and used according to the -manufacturer's recommendation. Cells (10⁶/aliquot) were re-suspended in PBS containing 3% BSA and 0.1% odium azide (FACS-buffer) and incubated with fluorescently labeled antibodies for 30 minutes (4° C.) followed by two washes in FACS buffer. Fluorescence data were acquired on a FACSCalibur® flow cytometer (Becton, Dickinson, Mountainview, Calif.) and analyzed using CELLQuest software (Becton Dickinson). This experiment was repeated once with similar results.

Analysis of cytokine production. Cytokine production was measured by analysis of culture supernatants by sandwich ELISA using murine IFNγ, TNFα, IL-2, and IL-12 kits purchased from R&D Systems (Minneapolis, Minn.). The ELISA for murine MIF was performed as previously described (14). Inclusion of neutralizing anti-MIF mAb in the cultures complexes with biologically active MIF, rendering the MIF inactive but still detectable by later ELISA.

Tumor growth in vivo. Experiments to determine the effect of anti-MIF mAb on EG.7 tumor growth were performed in C57BL/6 mice following methods described previously (9). Cultured EG.7 cells were washed, resuspended in PBS, and 5×10⁶ cells (suspended in 0.1 ml of PBS) injected s.c. into the upper flank of mice (n=5 per group). Mice received an i.p. injection of 0.3 ml PBS, or IgG₁, isotype control antibody (0.5 mg), or purified anti-MIF mAb (0.5 mg) 1 h later and then every 24 h for 7 days. Tumor size was estimated on day 7 from orthogonal linear measurements made with Vernier calipers according to the formula: weight (mg)=[(width, mm)²×(length, mm)]/² (17). This experiment was repeated twice with similar results.

Histologic studies. Tumors from control IgG and anti-MIF treated mice were excised at 7 days. Frozen tumor sections were stained using PE-CD4 (L3T4) and FITC-CD8 (Ly-2) mAbs (PharMingen). The CD8⁺ and CD4⁺ T cells were counted under a fluorescence microscope and expressed as % increase in the mean number of stained cells per tumor section compared to sections from the control IgG-treated mice. Ten fields per section were counted using a 10× objective (n=5 mice per group). Control sections incubated with a fluorescent-conjugated isotype control antibody showed no immunoreactivity.

In situ apoptosis detection. Cells undergoing apoptosis were detected using terminal deoxynucleotidyl transferase (TdT)-mediated dUTP-biotin nick end labeling (TUNEL) according to the manufacturer's recommended procedure (R&D Systems). For statistical analysis, apoptotic cells were counted by light microscopy (100×) and expressed as the mean number (±S.D.) of apoptotic cells per tumor section. Five random fields per section (1 section per mouse, 5 mice per group) were analyzed and the Student's t test was used to determine significance (p<0.05).

In vivo lymphoid cell migration assay. Non-tumor bearing mice or mice bearing EG.7 tumors of similar size (,) 7 days after tumor cell injection), as described previously by Zou et al. (18), treated with daily injections of anti-MIF (0.5 mg/mouse, i.p.) or control IgG, were used as the source of cells for this assay. Unfractionated spleen cells or purified splenic CD8⁺ T cells (1×10⁶ cells/ml) were obtained and labeled with PKH-26, a membrane-inserting red fluorescent dye (Sigma, St. Louis, Mo.). In vivo lymphoid migration assays were performed as previously described (n=5 mice per group) (19). Briefly, labeled cells were injected (i.v.) into tumor-bearing recipient mice. Tumor masses were removed 24 h later and cryostat sections were repared. Sections were stained with FITC-anti-CD4 or FITC-anti-CD8 to determine T cell type. The presence of PKH-26 fluorescent donor cells was quantified by microscopy and expressed as the mean number of labeled donor cells per field of sectioned tumor tissue. For each section (1 per mouse), ten fields were enumerated using a 10× objective. These experiments were repeated twice with similar results.

Adoptive Immunotherapy. C57BL/6 mice were injected with 5×10⁶ EG.7 cells (s.c) and then treated with anti-MIF mAb or control IgG (0.5 mg/day, i.p.) daily for 7 days (n=5 per group). One day after the last injection, spleen cells were isolated and CD8⁺ splenic T cells were purified using CD8⁺ enrichment colunms (R&D Systems). Unfractionated splenocytes or CD8⁺ T cells (5×10⁶ 3 cells/mouse) were then transferred (i.v.) into recipient mice that had been injected with 5×10⁶ EG.7 cells (i.p.) one day earlier. Tumor weights were determined on days 1-13 as described above. This experiment was repeated once with similar results.

Results

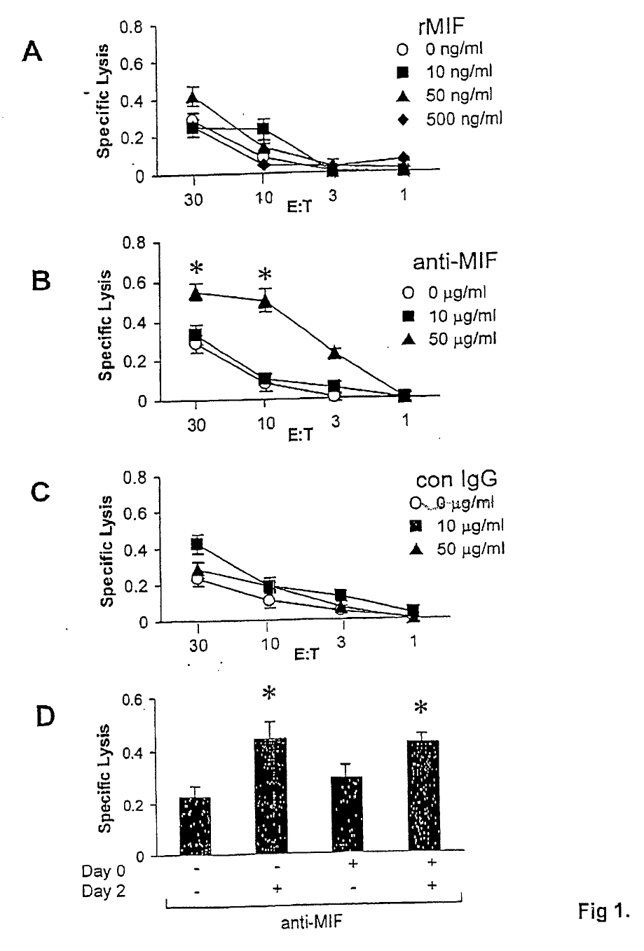

Anti-MIF antibody enhances CTL activity in vitro. Previous studies established that MIF protein and MRNA are expressed as part of the macrophage and the T lymphocyte activation response (5, 8, 20). To evaluate a potential role for MIF in the host response to tumor invasion, the inventors first examined whether rMIF or a neutralizing anti-MIF mAb influenced antigen-specific, cytotoxic T cell responses in vitro. Splenocytes from mice primed by the implantation of EG.7 cells were isolated, and these spleen cell cultures were stimulated for 5 days with irradiated EG.7 cells in the presence of either rMIF, neutralizing anti-MIF mAb, or isotype control IgG. As shown in FIG. 1B, the addition of anti-MIF mAb at 50 μg/ml significantly up-regulated the in vitro CTL response, whereas addition of exogenous rMIF (FIG. 1A) or control IgG (FIG. 1C) did not affect CTL activity. Control studies showed that anti-MIF mAb treatment of splenocytes or EG.7 cells alone did not influence their survival or growth characteristics, and that in vitro pre-treatment with anti-MIF mAb did not independently cause the development of cytotoxicity in unconditioned splenocyte cultures.

A potential role for MIF in the effector phase of the CTL response was also studied by adding anti-MIF mAb or rMIF during the final 4 h assay period of splenocyte culture with EG.7 target cells. There was no effect of these agents on the in vitro cytotoxic activity during this assay period. By contrast, it was observed that anti-MIF mAb was most active in augmenting the CTL response in vitro when present within the first two days of the five day co-culture period (FIG. 1D).

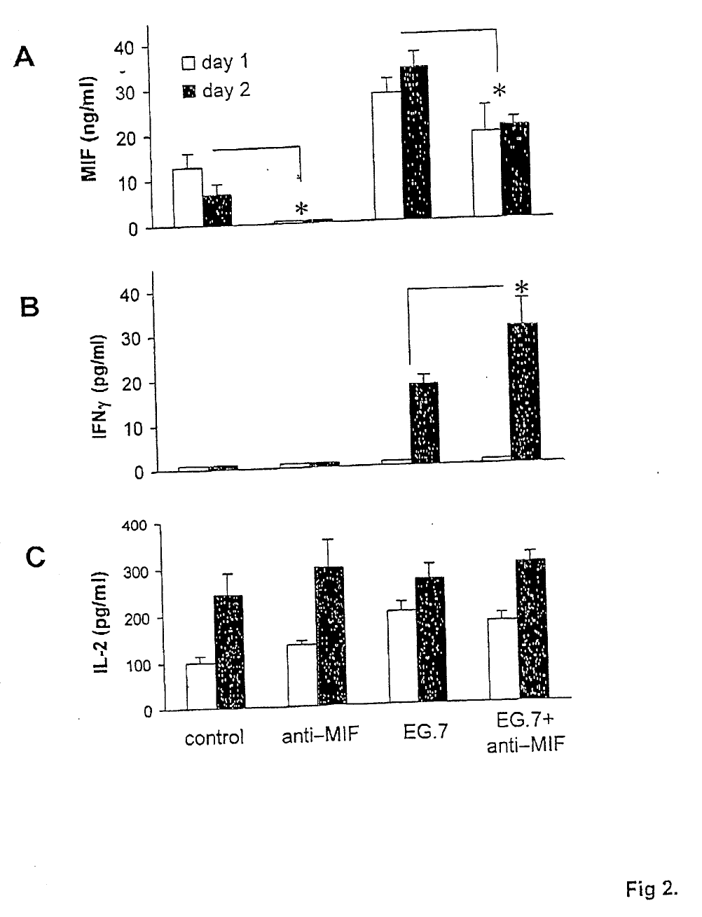

These data indicate that the immunoneutralization of MIF during the early phase of cytotoxic T cell activation in vitro potentiates later CTL activity. Not unexpectedly, therefore, it was found that in vitro stimulation of splenocyte effector cells with irradiated EG.7 target cells produced a significant increase in the amount of MIF detectable in culture supernatant when compared to splenocytes obtained from tumor-bearing mice cultured in the absence of irradiated EG.7 cells (FIG. 2A). Nevertheless, no significant effect on CTL activity was observed following the addition of bioactive, rMIF to parallel splenocyte cultures, suggesting that there may already exist a maximum cellular response to MIF that is endogenously produced in these cultures (>30 ng/ml) (FIG. 1A).

Next the effect of immunoneutralization of MIF on production of cytokines known to play an important role in the expression of T cell cytotoxicity in vitro was examined. Levels of IFNγ, TNFα, IL-2, and IL-12 present in the culture supernatants were measured by specfic ELISA. Among these, only IFNγ showed a significant increase in concentration during the two day co-culture period when anti-MIF mAb is most active in enhancing CTL activity when compared to the control mAb-treated cultures (FIG. 2B). By contrast, incubation of splenocyte cultures from EG.7 tumor-bearing mice in the presence of anti-MIF mAb and irradiated EG.7 cells did not significantly alter IL-2, IL-12, or TNFα protein expression when compared to control IgG treated cultures. EG.7 cells cultured alone revealed no detectable levels of MIF, IFNγ, IL-2, TNFα, or IL-12. By flow cytometric analysis, neither rMIF nor anti-MIF treatment of co-cultures wase found to influence the percentage of cells displaying the cell surface markers CD3⁺, CD4⁺, CD8⁺, CD28⁺, CD44^{high}.

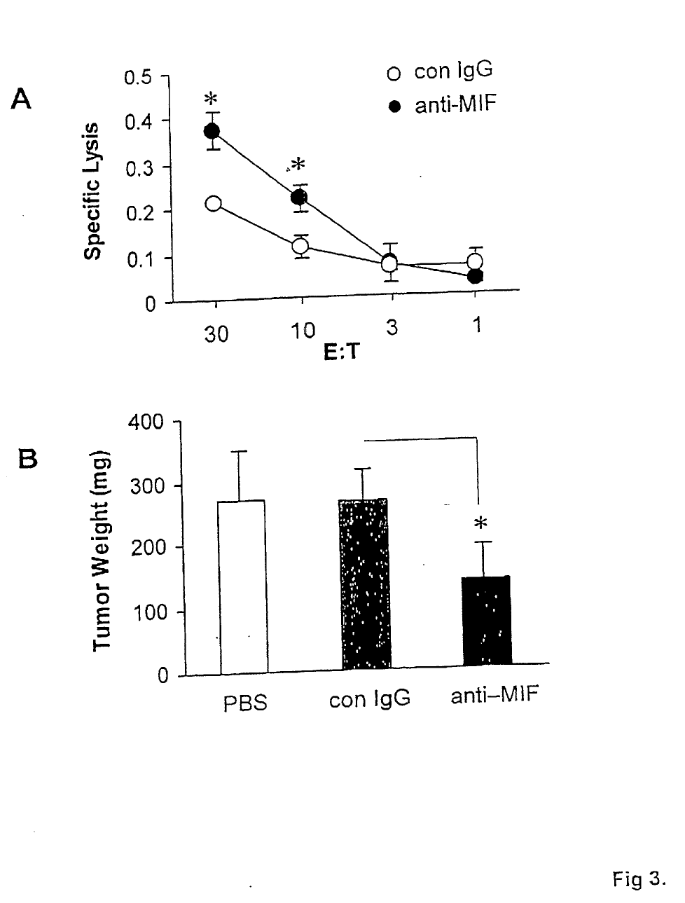

Anti-MIF mAb treatment in vivo enhances CTL activity. The CTL response of splenocytes harvested from mice treated with anti-MIF mAb versus an isotype control IgG, were compared next, during the period of EG.7 tumor priming in vivo. These experiments showed that the administration of anti-MIF mAb daily for one week after priming with EG.7 cells (on day 0) significantly enhanced the generation of CTL activity at E:T ratios of 30 and 10 (FIG. 3A). Inclusion of control IgG did not lead to enhanced CTL activity in this experimental system whether compared to either PBS alone or to no addition.

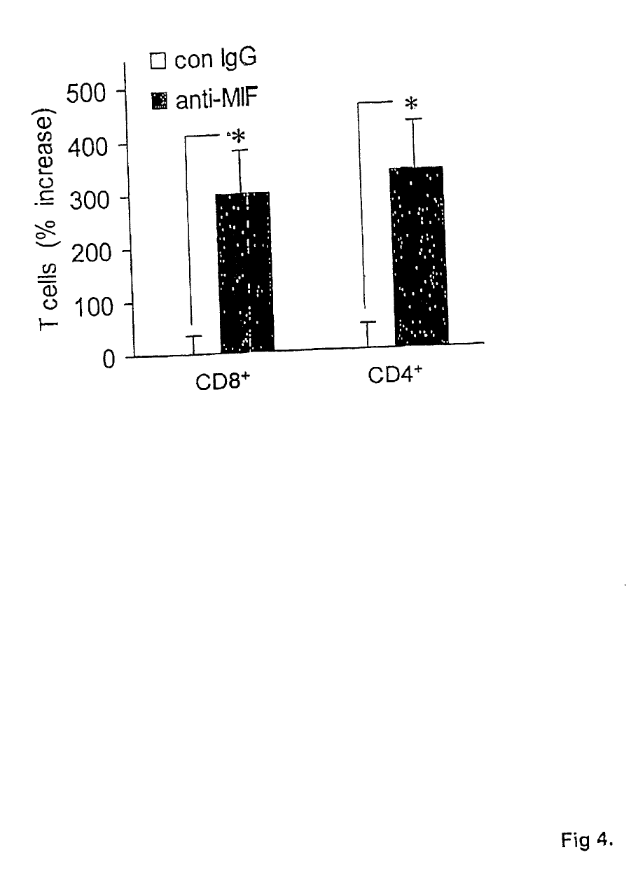

Recent studies have established a significant anti-tumor effect of anti-MIF mAb in mice bearing the 38C13 B cell lymphoma (9) and the G361 melanoma (10). In accordance with these data and the observed two-fold enhancement of CTL activity by anti-MIF described above, we found that administration of anti-MIF mAb to mice bearing an EG.7 lymphoma tumor for one week also resulted in a significant two-fold reduction in tumor size when compared to control IgG or PBS treated mice (FIG. 3B). In addition, we detected approximately three-fold more tumor infiltrating CD8⁺ and CD4⁺ cells following anti-MIF mAb treatment (FIG. 4).

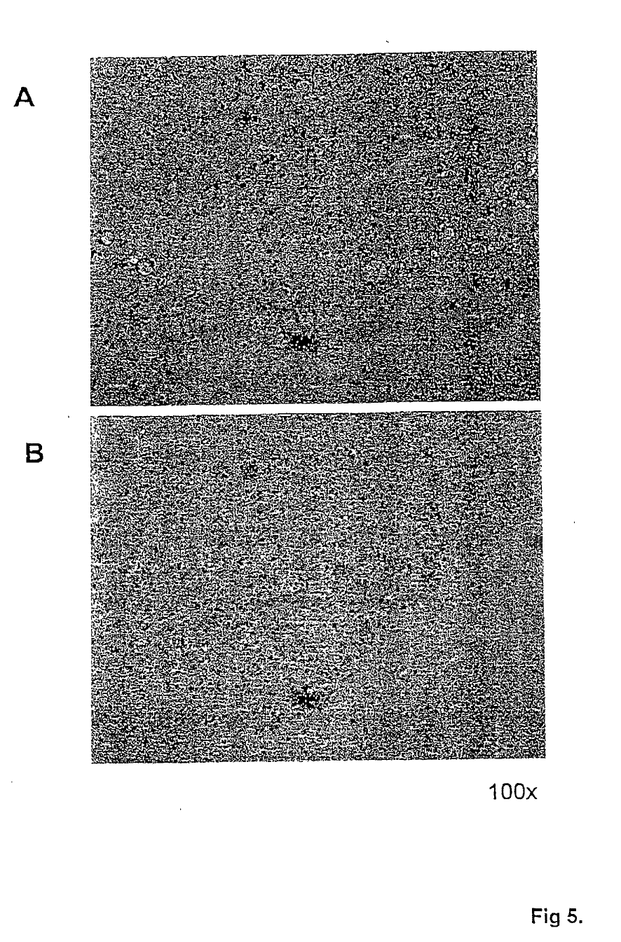

Cytotoxic T lymphocytes kill tumor cell targets by inducing apoptosis (21). Consistent with the observed enhancement of host CTL activity by anti-MIF treatment, a significant increase (4-5 fold) in the number of apoptotic cells within the tumor masses obtained from the anti-MIF-treated mice was found (FIG. 5A), compared to tumors obtained from control IgG treated mice (FIG. 5B). This difference in apoptosis was quantified by analyzing the average number of apoptotic cells per high power field in tumor sections from anti-MIF treated mice (194±63 cells /100× field) vs. the number from control IgG treated mice (43±22 cells/100× field) and found to be statistically significant (p<0.01).

It was previously reported that rMIF inhibits NK cell activity in vitro (22). Accordingly, the inhibitory effect of anti-MIF mAb on tumor growth in vivo might be the result of enhanced NK cell activity. While an increase in the NK activity of whole spleen cell preparations from EG.7 bearing mice when compared to control, non-tumor bearing mice, was observed, there were no changes observed in this activity in mice treated with anti-MIF antibodies.

Prior studies showed that MIF expression during antigen-driven CD4⁺T cell activation in vivo plays an important role in the immune response (8). Therefore, it was next determined whether the enhanced cytolytic activity observed with anti-MIF mAb treatment was associated with increased antigen-induced proliferation of CD8⁺ T cells. In accordance with Bacher et al. (8), no augmentation in T cell proliferation was found in the presence of anti-MIF mAb treatment in vivo.

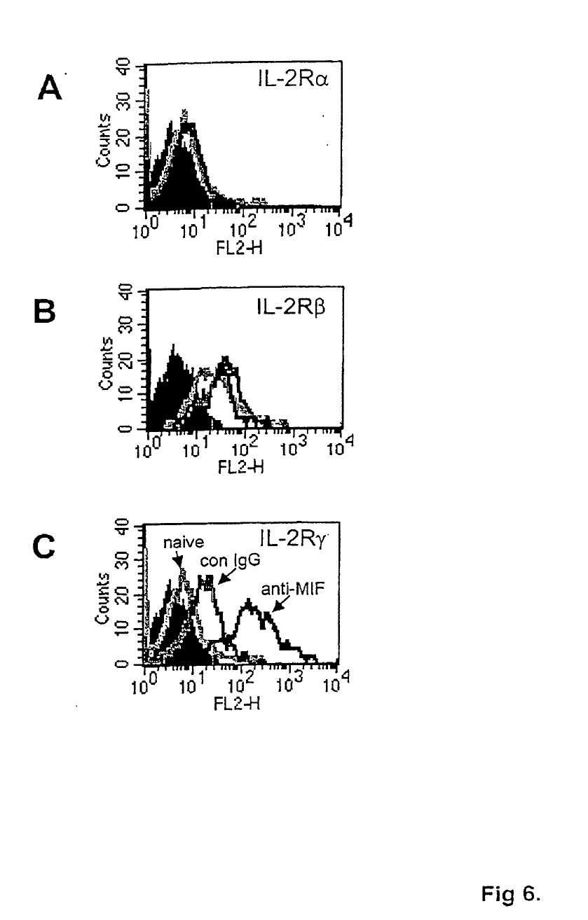

The effect of anti-MIF on IL-2 receptor expression also was examined. The IL-2 receptor is multimeric, consisting of the variably expressed a chain (CD25) which regulates IL-2 affinity, as well as two signaling subunits, the β (CD122) and the γ_{c}, (CD132) chains (reviewed in (23)). The γ_{c′} subunit (also known as the common gamma chain) is a shared subunit of the IL-2, IL-4, IL-7, IL-9, and the IL-15 receptors. Recruitment of the γ_{c}, is required for intracellular signaling (24, 25), and its expression has been shown to be critical for mature CD8⁺ T cell survival in vivo (26). Therefore, the effect of anti-MIF treatment on γ_{c} expression was examined. Anti-MIF mAb treatment of tumor-bearing mice significantly enhanced expression of the γ_{c} chain, but not of the α or β subunits of the IL-2 receptor on CD8⁺ T cells (FIG. 6), when compared to tumor-bearing animals treated with control IgG.

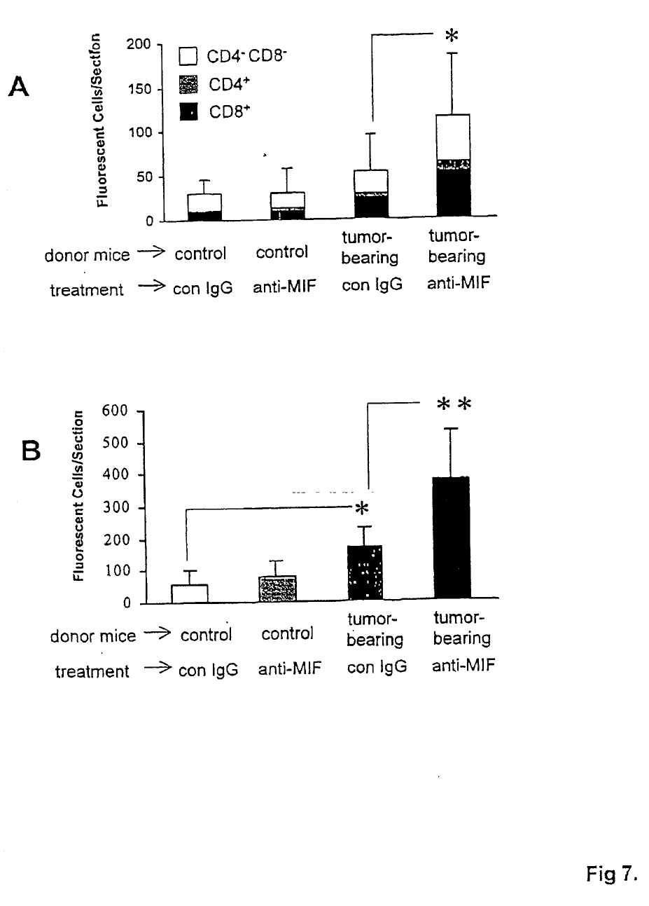

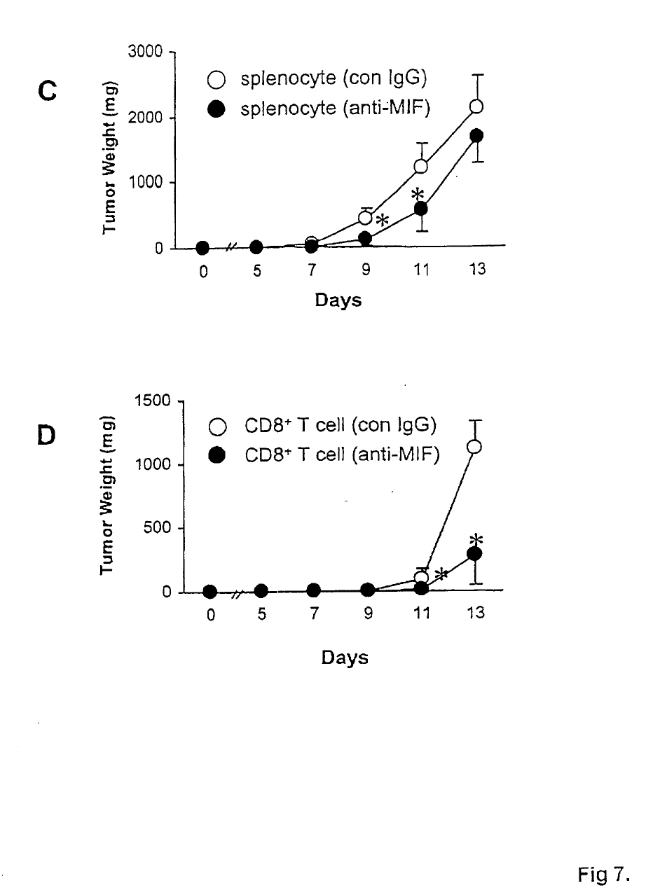

Anti-MIF antibody promotes the migration of T lymphocytes into tumor tissue and augments CD8⁺ T cell specific anti-tumor activity. To further show that the in vivo anti-tumor effect of anti-MIF mAb was attributable to specific effects on T cells, the effects of anti-MIF treatment on trafficking of T lymphocytes into tumors was assessed. Control or EG.7 tumor-bearing mice were treated with either anti-MIF or control IgG for 7 days, and unfractionated spleen cells or purified splenic CD8⁺ T cells were collected for labeling with PKH-26. Labeled unfractionated splenocytes or purified CD8⁺ cells were transferred into EG.7 tumor-bearing recipients. The entry of PKH-26- labeled donor cells into tumors of recipient mice over 24 hrs was quantified by fluorescent microscopy of cryostat sections obtained from excised tumor tissue (FIGS. 7A and 7B, respectively). These experiments showed that spleen cells or purified CD8⁺ T cells obtained from the anti-MIF mAb-treated, tumor-bearing mice entered tumor tissue in greater numbers (≧two-fold increase) than comparable cells obtained from the control mAb-treated, tumor-bearing mice.

Finally, the effect of adoptively transferred CD8⁺ cells (obtained from anti-MIF mAb treated animals) on tumor growth in vivo was tested. Five million unfractionated splenocytes or purified CD8⁺ splenic T cells (FIG. 7C) from anti-MIF mAb or control IgG treated, EG.7 tumor-bearing donor mice were transferred to mice that had been injected s.c. with EG.7 tumor cells 24 h previously. Tumor growth in vivo then was monitored for 2 weeks. As shown in FIG. 7C, adoptive transfer of CD8⁺ T cells obtained from anti-MIF-treated, tumor-bearing mice to untreated tumor-bearing mice showed a significant inhibitory effect on subsequent tumor outgrowth in recipient mice. In contrast, no significant difference in tumor weight was observed following the transfer of unfractionated splenocytes (5×10⁶ cells; containing both CD4⁺ and CD8⁺ T cells and B cells) obtained from anti-MIF treated vs. control IgG tumor-bearing mice. These data indicate the importance of a critical number of CD8⁺ T cells obtained from anti-MIF treated tumor-bearing animals to mediate a significant inhibition of tumor growth in adoptive transfer experiments.

The invention having now been fully described with reference to representative embodiments and details, it will be apparent to one of ordinary skill in the art that changes and modifications can be made thereto without departing from the spirit or scope of the invention as set forth herein.

### REFERENCES

1. Metz, et al., “Role of Macrophage Migration Inhibitory Factor in the Regulation of the Immune Response” Adv.Immunol., 66, 197-223 (1997).

2. Bloom, et al., “Mechanisms of a Reaction in vitro Associated with Delayed-Type Hypersensitivity”, Science, 111, 514. (1966).

3. David, “Delayed-Type Hypersensitivity in vitro: its Mediation by Cell-Free Substances Formed by Lymphoid-Antigen Interaction”, Proc.Natl.Acad.Sci. U.S.A., 56, 72 (1966).

4. Bernbagen, et al., “MEP is a Pituitaxy-Derived Cytokine that Potentiates Lethal Endotoxaemia, Nature 365, 756 (1993) [published erratum appears in Nature Nov. 23, 1995 378(6555):419].

5. Calandra, et al., “The Macrophage is an Important and Previously Unrecognized Source of Macrophage Migration Inhibitory Factor”, J. Exp. Med., 179, 1895 (1994).

6. Calandra, et al., “Macrophage Migation Inhibitory Factor is a Critical Mediator of the Activation of Immune Cells by Exotoxins of Gram-Positive Bacteria”, Proc. Nat'l. Acad. Sci. U.S.A,. 95, 11383 (1998).

7. Calandra, et al., “Protection From Septic Shock by Neutralization of Macrophage Migration Inhibitory Factor”, Nat. Med., 6, 164 (2000).

8. Bacher, et al., “An Essential Regulatory Role for Macrophage Migration Inhibito Factor in T-Cell Activation, Proc.Natl. Acad.Sci. U.S.A., 93, 7849 (1996).

9. Chesney, et al., “An Essential Role for Macrophage Migration Inhibitory Factor (MIF) in Angiogenesis and the Growth of a Murine Lymphoma”, Mol. Med., 5, 181 (1999).

10. Shimizu, et al., “High Expression of Macrophage Migration Inhibitory Factor in Human Melanoma Cells and its Role in Tumor Cell Growth and Angiogenesis”, Biochem. Biophys. Res. Commun., 264, 75 1 (1999).

11. Moore, et al., “Introduction of Soluble Protein into the Class I Pathway of Antigen Processing and Presentation”, Cell, 54, 777 (1988).

12. Bendrat, et al., “Biochemical and Mutational Investigations of the Enzymatic Activity of Macrophage Migration Inhibitory Factor”, Biochemistry 36, 15356 (1997).

13. Bernhagen, et al., “Purification, Bioactivity, and Secondary Structure Analysis of Mouse and Human Macrophage Migration Inhibitory Factor (MIF)”, Biochemistry, 33, 14144 (1994).

14. Calandra, et al., “MIF as a Glueocorticoid-induced Modulator of Cytokine Production”, Nature, 377, 68 (1995).

15. Ke, et al., “Ovalbumin Injected with Complete Freund's Adjuvant Stimulates Cytolytic Responses”, Eur. J. Immunol., 25, 549 (1995).

16. Hashimoto, et al., “Differential Antitumor Effects of Administration of Recombinant IL-18 or Recombinant IL-12 Are Mediated Primarily by Fas-Fas Ligand- and Perforin-Induced Tumor Apoptosis, Respectively”, J. Immunol., 163:583 (1999).

17. Taetle, et al., “Use of Nude Mouse Xenografts as Preclinical Drug Screens: in Vivo Activity of Established Chemotherap Agents Against Melanoma and Ovarian Carcinoma Xenografts”, Cancer Treat.Rep., 71, 297 (1987).

18. Zou, et al., “Tumor-Bearing Mice Exhibit a Progressive Increase in Tumor Antigen-Presenting Cell Function and a Reciprocal Decrease in Tumor Antigen-Responsive CD4⁺ T Cell Activity”, J. Immunol., 148:648 (1992).

19. Rosenblatt-Velin, et al., “Transformed and Nontransformed Human T Lymphocytes Migrate to Skin in a Chimeric Human Skin/SCID Mouse Model”, J. Invest.Dermatol., 109, 744 (1997).

20. Bernbagen, J., et al., “An Essential Role for Macrophage Migration Inhibitory Factor in the Tuberculin Delayed-Type Hypersensitivity Reaction”, J. Exp. Med 183:277 (1996).

21. Berke, G. et al., “The CTL's Kiss of Death”, Cell, 81, 9 (1995).

22. Apte, et al., “Role of Macrophage Migration Inhibitory Factor in Inhibiting NK Cell Activity and Preserving Immune Privilege” J. Immunol., 160, 5693 (1998).

23. Nelson, et al., “Biology of the Interleukin-2 Receptor”, Adv. Immunol., 70, 1-81, 1 (1998).

24. Nelson, et al., “A Membrane-proximal Region of the Interleukin-2 Receptor Gamma C Chain Sufficient for Jak Kinase Activation and Induction of Proliferation in T Cells”, Mol. Cell Biol., 16, 309 (1996).

25. Nelson, et al., “Cytoplasmic Domains of the Interleukin-2 Receptor Beta and Gamma Chains Mediate the Signal for T-cell Proliferation”, Nature, 369, 333 (1994).

26. Dai, et al., “The Role of the Common Cytokine Receptor Gamma-chain in Regulating IL1-2-Dependent, Activation Induced CD8⁺ T Cell Death”, J. Immunol., 163, 3131 (1999).

27. Jager, E., et al., “CTL-Defined Cancer Vaccines: Perspectives for Active Immunotherapeutic Interventions in Minimal Residual Disease”, Cancer Metastasis Rev., 18, 143 (1999).

28. Dunbar, et al., “Cutting Edge: Rapid Cloning of Tumor-specific CTL Suitable for Adoptive Immunotherapy of Melanoma”, J. Immunol., 162, 6959 (1999).

29. Thurner, B., et al., “Vaccination with Mage-3A1 Peptide-Pulsed Mature, Monocyte-Derived Dendritic Cells Expands Specific Cytotoxic T Cells and Induces Regression of Some Metastases in Advanced Stage IV Melanoma”, J. Exp. Med., 190:1669 (1999).

30. Yamasaki, et al., “Immunoregulatory Effects of Interleukin 2 and Interferon on Syngeneic Murine Malignant Glioma-Specific Cytotoxic T-Lymphocytes”, Cancer Res., 48, :2981 (1988).

31. Renauld, et al., “Accessory Signals Immurinecytolytic T Cell Responses. Dual Requirement for IL-1 and IL-6”, J. Immunol., 143, 1894 (1989).

32. Smyth, et al., “IL-2 and IL-6 Synergize to Augment the Pore-Forming Protein Gene Expression and Cytotoxic Potential of Human Peripheral Blood T Cells”, J. Immunol., 145, 1159 (1990).

33. Kasper, et al., “IL-7 Stimulates Protective Immunity in Mice Against the Intracellular Pathogen, Toxoplasma gondii”, J. Immunol., 155, 4798 (1995).

34. Chen, et al., “IL-10: a Novel Cytotoxic T Cell Differentiation Factor”, J. Immunol., 147, 528 (1991).

35. Gately, et al., “Administration of Recombinant IL-12 to Normal Mice Enhances Cytolytic Lymphocyte Activity and Induces Production of IFN-gamma in vivo”, Int. Immunol, 6, 157 (1994).

36. Gately, et al., “Regulation of Human Cytolytic Lymphocyte Responses by Interleukin-12”, Cell Immunol., 143:127 (1992).

37. Mehrotra, et al., “Effects of IL-12 on the Generation of Cytotoxic Activity in Human CD8+ T Lymphocytes”, J. Immunol., 151, 2444 (1993).

38. Ciolli, et al., “Combined Interieukin 1/interleukin 2 Therapy of Mice Injected with Highly Metastatic Friend Leukemia Cells: Host Antitumor Mechanisms and Marked Effects on Established Metastases”, J. Exp. Med., 173:313 (1991).

39. Rosenberg, et al., “Regression of Established Pulmonary Metastases and Subcutaneous Tumor Mediated by the Systemic Administration of High-Dose Recombinant Interleukin 2”, J. Exp. Med., 161, 1169 (1985).

40. Nastala, et al., “Recombinant IL-12 Administration Induces Tumor Regression in Association with IFN Gamma Production”, J. Immunol., 153:1697 (1994).

41. Hashimoto, et al., “Cytotoxic NK1.1 Ag+ Alpha Beta T cells with Intermediate TCR Induced in the Liver of Mice by IL-12”, J. Immunol., 154:4333 (1995).

42. Brunda, et al., “Interleukin-12: Murine Models of a Potent Antitumor Agent”, Ann. N. Y Acad. Sci., 795, 266-274, 266 (1996).

43. Brunda, et al., “The Anti-tumor Effect of Recombinant Interferon Alpha or Gamma is Influenced by TumorLIocation”, Int. J. Cancer, 40, :807 (1987).

44. Sayers, et al., “Antitumor Effects of Alpha-Interferon and Gamma-Interferon on a Murine Renal Cancer (Renca) in vitro and in vivo”. Cancer Res., 50, 5414 (1990).

45. Giovarelli, et al., “Interferon-Activated Tumor Inhibition in vivo. Small Amounts of Interferon-Gamma Inhibit Tumor Growth by Eliciting Host Systemic Immunoreactivity”, Int. J. Cancer, 37, 141 (1986).

46. Mule, et al., “Antitumor Effect of Recombinant Tumor Necrosis Factor-alpha Against Murine Sarcomas at Visceral Sites: Tumor Size Influences the Response to Therapy”, Cancer Immunol. Immunother., 26, 202 (1988).

47. Good, et al., “IL-2 and IL-4 Can Co-Modulate the Generation of Cytotoxic T Cells Through CD8- CD4- Splenic Lymphocytes”, Immunology, 67:225 (1989).

48. Yamashita, et al., “Suppressive Activity of Interleukin 4 on Tie Induction of Antigen-Specific Cytotoxic T Cells in Humans”, Jpn. J. Cancer Res., 82, 585 (1991).

49. Jin, et al., “TGf Beta Down-Regulates TLiSA1 Expression and Inhibits the Differentiation of Precursor Lymphocytes into CTL and LAK cells”, Immunology 66, 570 (1989).

50. Erard, et al., “Switch of CD8 T Cells to Noncytolytic CD8- CD4- Cells that Make TH2 Cytokines and Help B Cells:, Science, 260, 1802 (1993).

51. Croft, et al., “Generation of Polarized Antigen-specific CD8 Effector Populations: Reciprocal Action of Interleukin (IL)-4 and IL-12 in Promoting Type 2 Versus Type 1 Cytokine Profiles”, J. Exp. Med., 180, 1715 (1994).

52. Noble, et al., “IFN-Gamma and IL-4 Regulate the Growth and Differentiation of CD8+ T cells into Subpopulations with Distinct Cytokine Profiles”, J. Immunol., 155, 2928 (1995).

53. Takahashi, et al., “Involvement of Macrophage Migration Inhibitory Factor (MIF) in the Mechanism of Tumor Cell Growth”, Mol. Med., 4, 707 (1998).

54. Meyer-Siegler, et al., “Enhanced Expression of Macrophage Migration Inhibitory Factor in Prostatic Adenocarcinoma Metastases”, Urology, 48, 448 (1996).

55. Rees, et al., “Selective MHC Expression in Tumours Modulates Adaptive and Innate Antitumour Responses” [see comments], Cancer Immunol. Immunother, 48, 374 (1999).

56. Hahne, et al., “Melanoma Cell Expression of Fas(Apo-1/CD95) Ligand: Implications for Tumor Immune Escape” [see comments], Science, 274, 1363 (1996).

57. Strand, et al., “Lymphocyte Apoptosis.Induced by CD95 (APO-I/Fas) Ligand-Expressing Tumor Cells—a Mechanism of Immune Evasion?” [see comments], Nat. Med., 2, 1361 (1996).

58. Chappell, et al., “Human Melanoma Cells do not Express Fas (Apo-I/CD95) Ligand”, Cancer Res., 59, 59 (1999).

59. Arai, et al., “Gene Transfer of Fas Ligand Induces Tumor Regression in vivo”, Proc. Nat'l. Acad. Sci. U.S.A., 94, 13862 (1997).

60. Kang, et al., “Fas Ligand Expression on Islets as well as Multiple Cell Lines Results in Accelerated Neutrophilic Rejection”, Transplant. Proc., 30, 538 (1998).

61. Seino, et al., “Contribution of Fas Ligand to Cardiac Allograft Rejection”, Int. Immunol., 8, 1347 (1996).

62. Seino, et al., “Rejection of Fas Ligand-Expressing Grafts”, Transplant. Proc., 29, 1092 (1997).

63. Allison, et al., “Transgenic Expression of CD95 Ligand on Islet Beta Cells Induces a Granulocytic Infiltration but Does Not Confer Immune Privilege upon Islet Allografts” [see comments], Proc. Nat'l. Acad. Sci. U.S.A, 94, 3943 (1997).

64. Liu, et al., “Suppressive Effect of Corticosteroids on the Gene Expression of Interleukin-5 and Eosinophil Activation in Asthmatics”, Chung. Hua. Nei. Ko. Tsa. Chih. 35, 231 (1996).

65. Thor, et al., “In situ T cells in Melanoma” [see comments]. Cancer Immunol. Immunother., 48, 386 (1999).

66. Hudson, et al., “A Proinflammatory Cytokine Inhibits p53 Tumor Suppressor Activity” [see comments]. J. Exp. Med., 190, 1375 (1999).

67. Matsumura, et al., “Emerging Principles for the Recognition of Peptide Antigens by MHC Class I Molecules” [see comments], Science, 257, 927 (1992).

What is claimed is:

1. A method of preparing cells as a cancer therapy for administration to a subject with cancer comprising culturing the cells in the presence of an MIF antagonist.

2. The method of claim 1 in which the MIF antagonist is selected from the group consisting of anti-MIF antibodies, MIF antisense cDNA, and antagonists of MIF ligand:receptor binding.

3. A method of preparing cells as a cancer therapy for administration to a subject with cancer comprising culturing the cells in the presence of anti-MIF antibodies.

4. The method of claim 3 in which the anti-MIF antibodies are monoclonal.

5. The method of claim 4 in which the monoclonal anti-MIF antibodies are selected from the group consisting of human monoclonal antibodies, humanized monoclonal antibodies, chimeric monoclonal antibodies and single-chain monoclonal antibodies.

6. A method of preparing a cellular composition as a cancer therapy for administration to a subject with cancer comprising incubating cells of the composition in the presence of (a) at least one tumor antigen and (b) anti-MIF antibodies.

7. A method of preparing autologous cells for administration to a subject with cancer comprising the step of incubating the cells in the presence of an agent, said agent selected from the group consisting of anti-MIF antibodies, MIF-binding fragments thereof, or both.

8. A method of preparing autologous cells for administration to a subject with cancer comprising the step of incubating the cells in the presence of (a) at least one tumor antigen and (b) an agent, said agent selected from the group consisting of anti-MIF antibodies, MIF-binding fragments thereof, or both.

9. The method of claim 7 in which the autologous cells comprise immune cells.

10. The method of claim 7 in which the autologous cells comprise T cells.

11. The method of claim 7 in which the autologous cells comprise CD8⁺ cells.

12. A cellular composition for administration to a subject with cancer comprising cells incubated with anti-MIF antibodies.

13. The cellular composition of claim 12 in which the cells incubated with anti-MIF antibodies are also incubated with at least one tumor antigen.

14. The cellular composition of claim 12 in which the incubation with anti-MIF antibodies is ex vivo.

15. The cellular composition of claim 12 that is isolated from unbound anti-MIF antibodies.

16. The cellular composition of claim 13 that is isolated from unbound anti-MIF antibodies and unbound tumor antigen.

17. The cellular composition of claim 13 in which the cells comprise immune cells.

18. The cellular composition of claim 13 in which the cells comprise T cells.

19. The cellular composition of claim 13 in which the cells comprise CD8⁺ cells.
